(19) United States
(12) Patent Application Publication
(10) Pub. No.: US 2007/0293811 A1
(43) Pub. Date: Dec. 20, 2007

(54) URINARY CATHETER STAND

(76) Inventor: Eugene Addington, Desert Hot Springs, CA (US)

Correspondence Address: BLACK LOWE & GRAHAM, PLLC, 701 FIFTH AVENUE, SUITE 4800, SEATTLE, WA, 98104, US

(21) Appl. No.: 11/611,513

(22) Filed: Dec. 15, 2006

Related U.S. Application Data
(60) Provisional application No. 60/804,750, filed on Jun. 14, 2006.

(51) Int. Cl. A61M 1/00; A47G 29/00

(52) U.S. Cl. 604/27; 248/80

(57) ABSTRACT

A urinary catheter drying stand includes a shelf unit. The shelf unit defines at least one scallop configured to vertically receive a urinary catheter in hanging engagement; and an attachment device for securing the shelf to a stationary object, thereby allowing the catheters to vertically hang to air dry.

### PRIORITY CLAIM

This application is related to and claims the benefit of U.S. Provisional Application 60/804,750 filed Jun. 14, 2006, the disclosure of which is incorporated herein by reference in its entirety.

### BACKGROUND OF THE INVENTION

Urinary bladder disease is a major medical problem in the United States and worldwide. It has been estimated that up to 35 million people suffer from some type of urinary bladder disease in the United States alone. Many of these individuals need to utilize urinary catheters on a regular, and even continuous, basis in order to empty their bladders.

Regular users of urinary catheters have an increased risk of developing urinary tract infections. In part, this occurs because the catheters are generally reusable and must be cleaned and dried before being used again. Drying is usually performed by laying the catheters on an absorbent material, such as a towel, until they dry. However, drying in this manner is not efficient and may result in the growth of microorganisms on the catheter which can cause urinary tract infections.

Therefore, there is a need for new apparatus for and methods of drying urinary catheters after cleaning.

### SUMMARY OF THE INVENTION

Urinary catheter drying stands and methods of drying urinary catheters using these stands are disclosed.

In one embodiment, the urinary catheter drying stand comprises a shelf unit defining one or more scallops each of which are adapted to grasp and vertically hang a urinary catheter and an attachment device for securing the shelf to a stationary object, thereby allowing the catheters to vertically hang.

In another embodiment, the urinary catheter drying stand comprises a shelf unit defining one or more scallops each of which are adapted to grasp and vertically hang a urinary catheter, a base unit having a bottom surface and a top surface and a shaft extending from the top surface of the base unit to the bottom surface of the shelf unit of sufficient length to allow the urinary catheters to vertically hang from the shelf unit.

In still another embodiment, the urinary catheter drying stand comprises an integrated case configured to envelop component parts of the urinary catheter stand when configured in the collected state. When deployed, clam shell halves of the case unfold to lock forming a base. The component parts are then assembled to form the urinary catheter drying stand.

### BRIEF DESCRIPTION OF THE DRAWINGS

FIG. 1 is an isometric view of an embodiment of a urinary catheter drying stand;

FIG. 2 is an exploded view of the embodiment of the urinary catheter drying stand;

FIG. 3 is an isometric view of an alternate embodiment of the urinary catheter drying stand in a deployed state;

FIG. 4 is a cross-sectional view of the alternate embodiment of the urinary catheter drying stand in the deployed state;

FIG. 5 is an isometric view of the alternate embodiment of the urinary catheter drying stand in a collected state;

FIG. 6 is a first-end cross-sectional view of the alternate embodiment of the catheter drying stand in the collected state;

FIG. 7 is a second-end cross-sectional view of the alternate embodiment of the catheter drying stand in the collected state; and

FIG. 8 is a third-end cross-sectional view of the alternate embodiment of the catheter drying stand in the collected state.

### DETAILED DESCRIPTION OF THE PREFERRED EMBODIMENT

The urinary catheter drying stand disclosed herein satisfies the need for new apparatus for and methods of drying urinary catheters after cleaning.

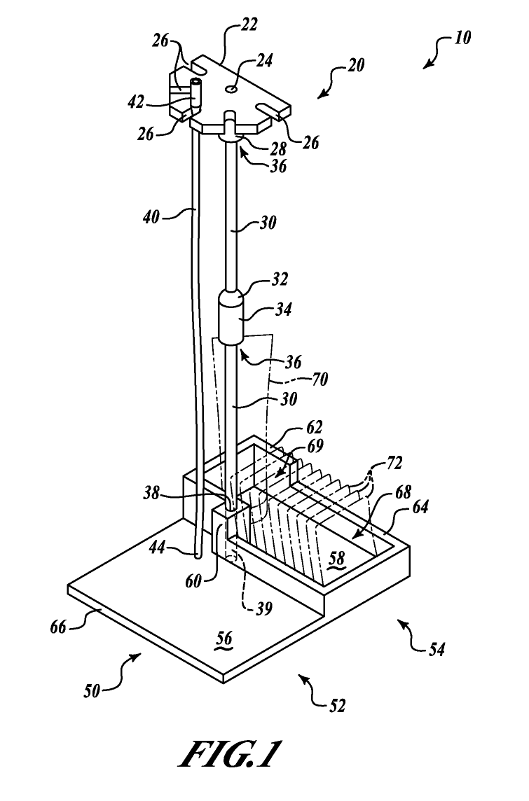

Referring now to FIG. 1, a urinary catheter drying stand 10 is shown that has a shelf unit 20 which includes a shelf plate 22 having a knuckle 28 defining a cavity 36 configured to receive a shaft 30 and a rivet 24 for securing the knuckle 28 to the shaft 30. The shaft 30, in this non-limiting exemplary embodiment, includes two shafts 30 joined at a ferrule 34 having a cavity 36 and a rounded shoulder 32.

The shelf unit 20 defines one or more scallops 26, in this instance five, each of which are adapted to grasp and vertically hang a urinary catheter 40 having a flange 42 and a tip 44. The knuckle 28 defining its cavity 36 comprises an attachment device. In an embodiment of the urinary catheter drying stand the shelf unit 22 defining the scallops 26 is configured to detachably attach to a stationary object such as a table or wall (not shown) in a manner which allows the urinary catheters to vertically hang for drying obviating the need for the shaft 30 and a base assembly 50. In such an embodiment, the attachment device facilitates the detachable attachment to the stationary object.

In an embodiment requiring the base assembly 50 for support of the shelf unit 20, the base assembly 50 includes a generally planar base plate 56 having a bead 66 surrounding the base plate 56 configured to prevent the escape of liquid draining from the catheter 40 while hanging from the scallop 26. The base plate 56 is further configured to receive the shaft 30 at an aperture 38 to a socket 39 the base plate defines. The socket 39 is configured to receive the shaft 30 in supporting engagement to elevate the shaft suitably above the base plate 56 to allow the catheter 40 to hang its full length vertically without contact with the base plate 56. In a nonlimiting embodiment, a land 60 is built up to strengthen the socket 39 by extending a depth of the socket 39. The base assembly 50 provides a stable and rugged base for the shelf unit 20 while supporting the catheter above the base plate 56.

The base plate 56 and the bead 66 are further configured to allow a folded facial tissue (not shown) to be placed under the hanging catheter 40 to absorb any liquid dripping from the catheter tip 44. To further facilitate the sterile drying of the catheter 40, the base plate 56, in a further non-limiting exemplary embodiment, includes a first section 52 and a second section 54 having a bulkhead 64 and a secondary bulkhead 62 which in combination with a deck 58 define a well 68 and a bay 69. The well 68 is configured to receive and hold a plurality of packaged moistened antiseptic towlettes 72 (shown in phantom). The bay 69 is configured to receive a tube 70 configured for dispensing a lubricant for insertion of the catheter 40 to facilitate venting of urine from a bladder.

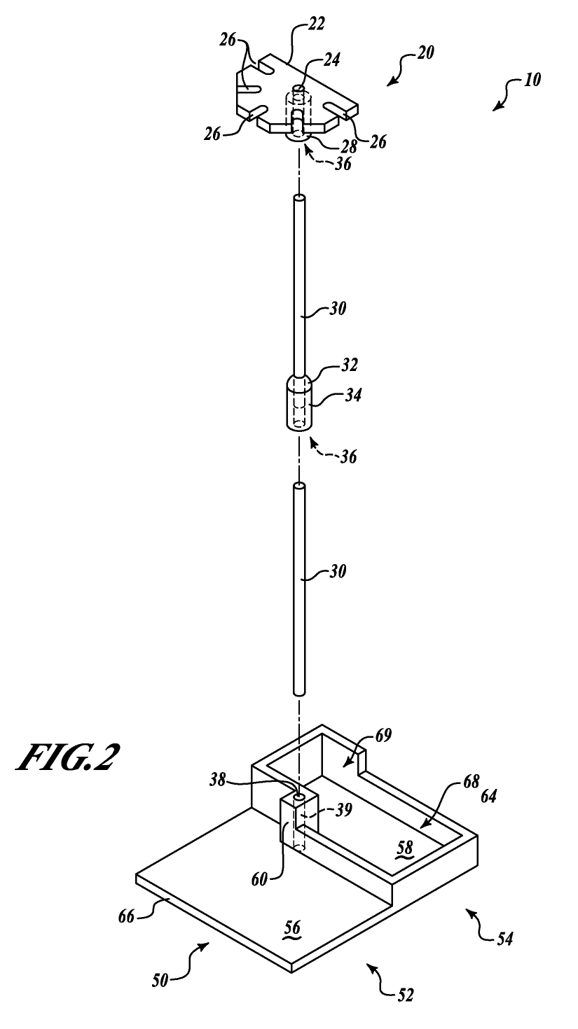

Referring now to FIG. 2, the urinary catheter drying stand 10 is shown in exploded view. As set forth above, the shelf unit 20 defining one or more scallops 26 includes the knuckle 28 defining the cavity 36 configured to receive the shaft 30 and the rivet 24 for securing the knuckle to the shelf unit 20. The shaft 30 is shown separated from the knuckle 28 to demonstrate the ready means by which the shaft is readily inserted into the knuckle. The two shafts 30 are readily disjointed at the ferrule 34 in a step preceding storage by removing the shaft 30 from the cavity 36 in the ferrule 34. The ferrule 34 is configured to have a rounded shoulder 32 to prevent pooling of liquid on an upper aspect of the ferrule 34. So configured, the ferrule 34 will not readily host the growth of bacteria.

The base assembly 50 at the base plate 56 is disjointed from the shaft 30 at the aperture 38 to the socket 39 the base plate 56 defines. The base 50 includes the land 60, the bead 66, the bulkhead 64, the deck 58 defining the well 68 and the bay 69. The base assembly 50 is generally static in disassembly further contributing inherent stability of the base assembly 50 in the pictured embodiment.

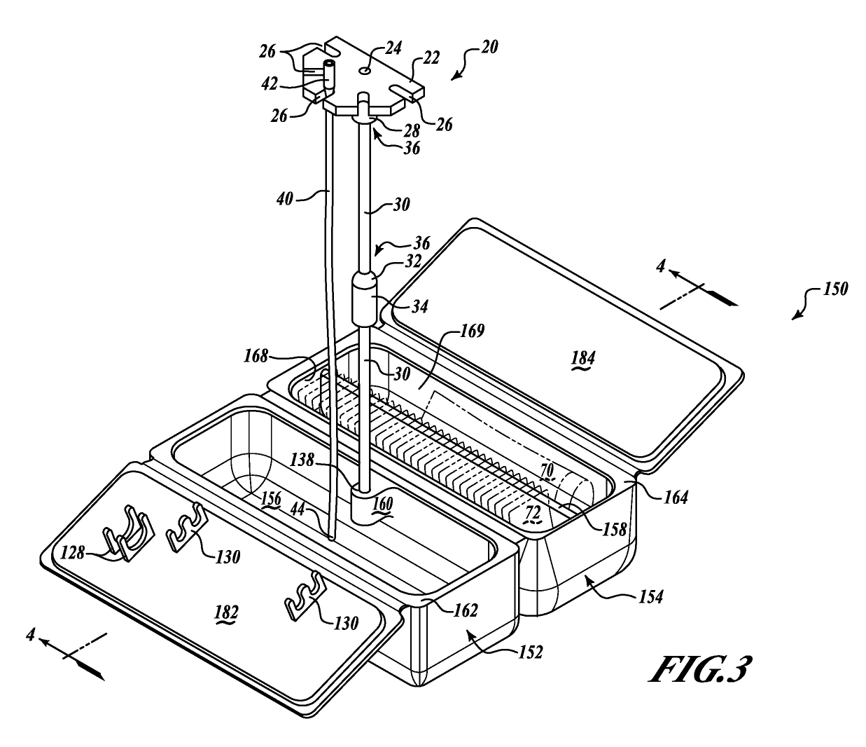

Referring now to FIG. 3, an embodiment of a urinary catheter drying stand 100 wherein the base assembly 150 comprises an integrated case is shown in a deployed state. As set forth above, the shelf unit 20 includes the shelf plate 22 defining one or more scallops 26 and having the knuckle 28 defining the cavity 36 configured to receive the shaft 30 and the rivet 24 for securing the knuckle 28 to the shelf plate 22. As set forth above, the shaft 30 includes two shafts 30 joined at the ferrule 34 having the rounded shoulder 32 and the cavity 36. The urinary catheter 40 having the flange 42 and the tip 44 is hung vertically from the scallop 26.

In the embodiment shown in FIG. 3, the base assembly 150 comprises a first section 152 configured for support of the shelf unit 20 and shaft 30 which is hingedly connected to a second section 154 configured to store accessory supplies. The first section 152 includes a generally planar base plate 156 having a bulkhead 162 surrounding the base plate 156 configured to prevent escape of liquid draining from the catheter 40 while hanging from the scallop 26. The first section 152 is further configured to receive the shaft 30 at a socket 138 the bulkhead 162 defines. The socket 138 is configured to receive the shaft 30 in supporting engagement to elevate the shaft suitably above the base plate 156 to allow the catheter 40 to hang its full length vertically without contact with the base plate 156. In a nonlimiting embodiment, a land 160 is built up to strengthen the socket 138 by extending a depth of the socket 138. The first section further comprises hingedly connected lid 182 having a pair of fans of resilient fingers 130 configured to grasp the shafts 30 and to hold the shafts 30 in storage and a pair of U-shaped keepers 128 are also attached to the lid 182 and configured to hold and store the shelf unit 20. The second section 154 includes a bulkhead 164 which in combination with a deck 158 define a well 168 and a bay 169. The well 168 is configured to receive and hold a plurality of packaged moistened antiseptic towlettes 72 (shown in phantom). The bay 69 is configured to receive a tube 70 (shown in phantom) configured for dispensing a lubricant for insertion of the catheter 40 to facilitate venting of urine from a bladder. The second section is also hingably connected to a lid 184.

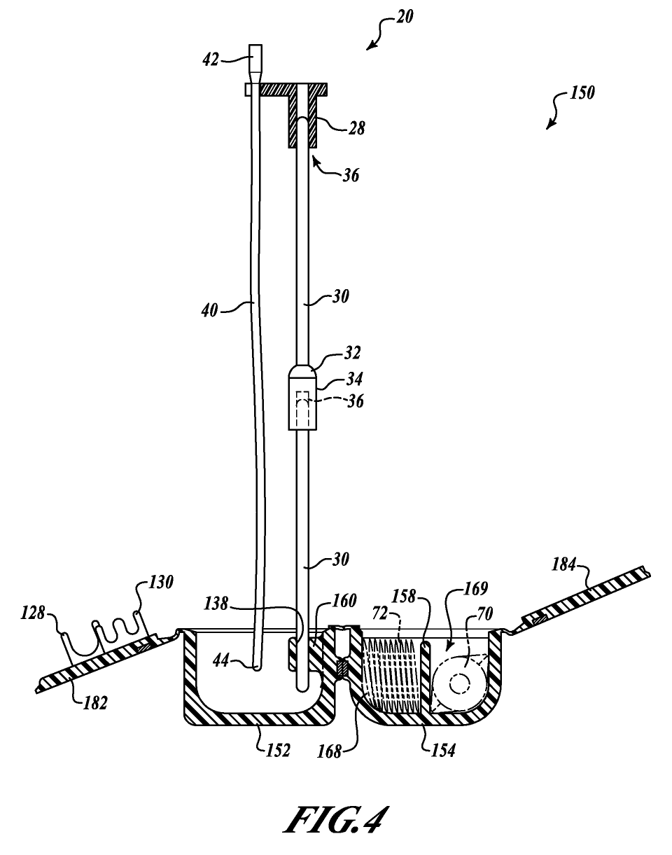

Referring now to FIG. 4, a cross-sectional view of the embodiment of a urinary catheter drying stand 100 comprising an integrated case is shown in a deployed state. As set forth above, the shelf unit 20 includes the generally planar shelf plate 22 having the knuckle 28 defining the cavity 36 configured to receive the shaft 30. The shaft 30 includes two shafts 30 joined at the ferrule 34 having the rounded shoulder 32 and the cavity 36. The base assembly 150 includes the first section 152 hingably connected to the second section 154. The first section 152 includes the socket 138 and the land 160 built to strengthen the socket 138 and is hingably connected to the lid 182 having the fans 130 configured to store the shafts 30 and the keepers 128 configured to store the shelf unit 20. The second section 154 includes the well 168 configured to receive and hold the plurality of packaged moistened antiseptic towlettes 72 (shown in phantom) and the bay 169 configured to receive the tube 70 (shown in phantom) configured for dispensing a lubricant for insertion of the catheter 40 to facilitate venting of urine from a bladder and is hingably connected to the lid 184. The urinary catheter 40 having the flange 42 and the tip 44 is hung vertically from the shelf unit 20.

Referring now to FIG. 5, the embodiment of a urinary catheter drying stand 100 comprising an integrated case is shown in a collected state. As set forth above, the shelf unit 20 includes the shelf plate 22 having the knuckle 28 defining the cavity 36 configured to receive the shaft 30. The shaft 30 includes two shafts 30 joined at the ferrule 34 having the cavity 36. The base assembly 150 includes the first section 152 hingably connected to the second section 154. The first section 152 includes the socket 138 and the land 160 built to strengthen the socket 138 and is hingably connected to the lid 182 having the fans 130 configured to store the shaft 30 and the keepers 128 configured to store the shelf unit 20.

To demonstrate the utility of the embodiment for containing and holding the shelf unit 20, the fans 128 have received the shafts 30 including the ferrule and keeper 130 is detachably securing the shelf unit 20 by resiliently encompassing the knuckle 28. The second section 154 includes the well 168 configured to receive and hold the plurality of packaged moistened antiseptic towlettes 72 (shown in phantom) and the bay 169 configured to receive the tube 70 (shown in phantom) configured for dispensing a lubricant for insertion of the catheter 40 to facilitate venting of urine from a bladder and is hingably connected to the lid 184. The urinary catheter 40 is shown stored in the first section 152 of the base assembly 150.

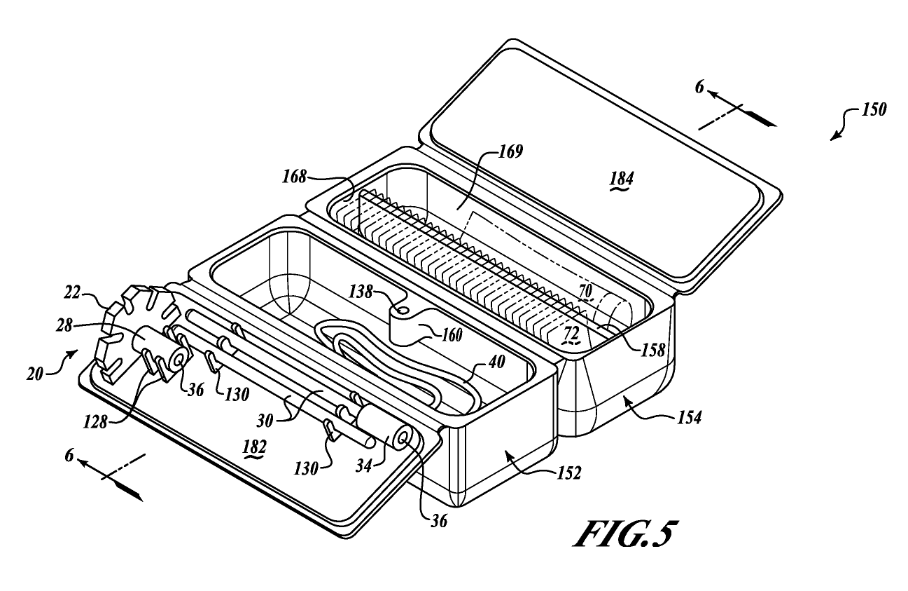

Referring now to FIGS. 6, 7 and 8, cross-sectional views of the embodiment of a urinary catheter drying stand 100 comprising an integrated case in a collected state and depicting closure of the integrated case are shown. In FIGS. 6, 7 and 8, as set forth above, the shelf unit 20 includes the shelf plate 22 having the knuckle 28 defining the cavity 36 configured to receive the shaft 30. The shaft 30 includes two shafts 30. The base assembly 150 includes the first section 152 hingably connected to the second section 154. The first section 152 includes the socket 138 and the land 160 built to strengthen the socket 138 and is hingably connected to the lid 182 having the keeper 130 configured to store the shaft 30 and the fans 128 configured to store the shelf unit 20. The second section 154 includes the well 168 configured to receive and hold the plurality of packaged moistened antiseptic towlettes 72 (shown in phantom) and the bay 169 configured to receive the tube 70 (shown in phantom) configured for dispensing a lubricant for insertion of the catheter 40 to facilitate venting of urine from a bladder and is hingably connected to the lid 184. The urinary catheter 40 is shown stored in the first section 152 of the base assembly 150.

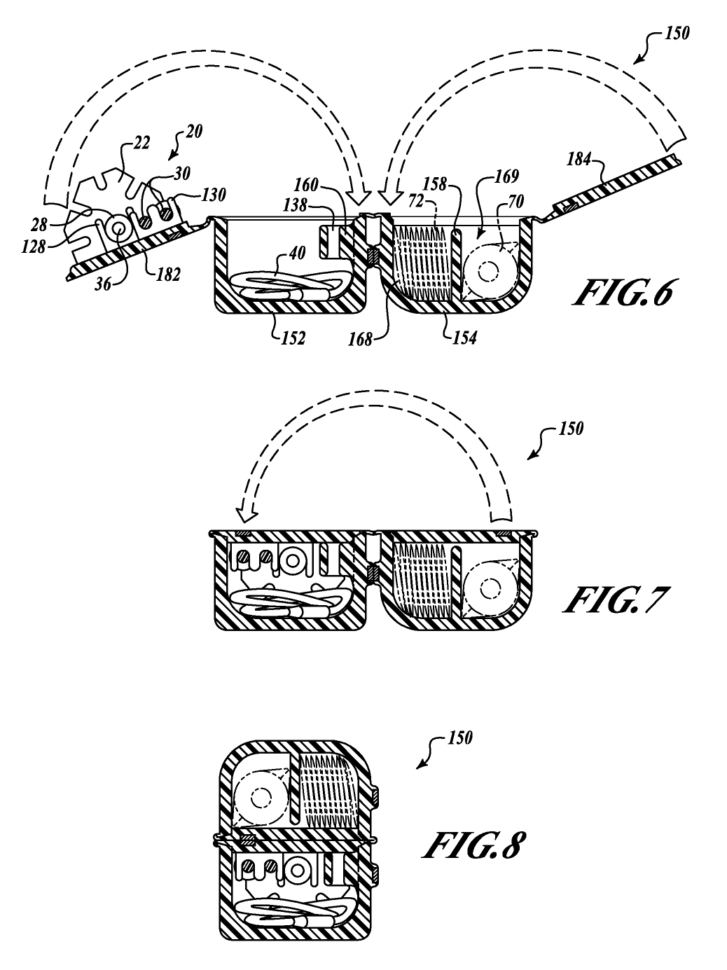

In order to close the urinary catheter drying stand 100 comprising an integrated case, the lid 182 hingably connected to the first section 152 of the base unit 150 is rotated clockwise to cover the first section 152 of the base unit 150 and the lid 184 hingably connected to the second section 154 of the base unit 150 is rotated counterclockwise to cover the second section 154 of the base unit 150 (FIGS. 6 and 7). The second section 154 of the base unit 150 hingably connected to the first section 152 of the base unit 150 is then rotated counterclockwise to cover the first section 152 of the base unit 150 (FIG. 8).

The urinary catheter drying stand disclosed herein can be fabricated from various types of readily available materials. In one embodiment, waterproof or water resistant materials such as plastic and stainless steel may be used. In another embodiment, the plastic used is Class 6 polypropylene Medical Grade Material. In yet another embodiment, the urinary catheter drying stand is fabricated from a material which renders the stand dishwasher safe.

The urinary catheter drying stand and methods of drying urinary catheters disclosed herein have certain advantages when compared to the previous method, which involves laying the catheters on an absorbent material, such as a towel, until they dry. A major advantage is that the drying is more efficient and complete and therefore less likely to allow the growth of infectious microorganisms when compared to the previous method, is part because less moisture gathers at the ends of the catheters when compared to the previous method. Another advantage is that the urinary catheter drying stand and methods of drying urinary catheters disclosed herein utilize less counter space than the previous method.

It will be understood that the present disclosure is not limited to the embodiments disclosed herein as such embodiments may vary somewhat. It is also to be understood that the terminology employed herein is used for the purpose of describing particular embodiments only and is not intended to be limiting in scope and that limitations are only provided by the appended claims and equivalents thereof.

The embodiments of the invention in which an exclusive property or privilege is claimed are defined as follows:

1. A urinary catheter drying stand comprising:
a shelf unit defining at least one scallop, wherein the scallop is configured to receive a urinary catheter in hanging engagement; and
an attachment device for securing the shelf to a stationary object, thereby allowing the catheters to vertically hang.

2. The stand according to claim 1, wherein the attachment device detachably attaches the shelf unit to a wall.

3. The stand according to claim 1, wherein the attachment device detachably attaches the shelf unit to a table.

4. The stand according to claim 1, wherein the attachment device includes a knuckle defining a cavity configured to receive a shaft.

5. The stand according to claim 4, further comprising:
the shaft; and
a base assembly including a base plate configured to receive the shaft at a socket the base assembly defines.

6. The stand according to claim 5, wherein the base assembly further defines:
a well to receive towlettes.

7. The stand according to claim 5, wherein the base assembly further defines a bay to receive tubed liquids.

8. The stand according to claim 5, wherein the base assembly further defines a bead to prevent the escape of liquid draining from a catheter.

9. The stand according to claim 5, wherein the base assembly further defines an integrated case.

10. A method for drying urinary catheters comprising vertically hanging at least one urinary catheters in the scallops of the shelf unit of the urinary catheter drying stand according to claim 1 and allowing the urinary catheters to dry.

11. A urinary catheter drying stand comprising:
(a) a shelf unit having at least one scallop, wherein the scallop is configured to receive a urinary catheter in hanging engagement;
(b) a base assembly; and
(c) a shaft which attaches to and extends from the base assembly to the shelf unit, attaches an attachment device on the shelf unit and is of sufficient length to allow the urinary catheter to vertically hang from the shelf unit.

12. The stand according to claim 11 wherein the shaft is one piece.

13. The stand according to claim 11 wherein the shaft is detachably attached to the base unit and the shelf unit.

14. The stand according to claim 13 wherein the shaft comprises two or more members detachably attached at one or more joints.

15. The stand according to claim 14 wherein the shaft comprises a first member and second member detachably attached at one joint.

16. The stand according to claim 11 wherein the base assembly further comprises a well to receive towlettes.

17. The stand according to claim 11 wherein the base assembly further comprises a bay to receive tubed liquids.

18. The stand according to claim 11, wherein the base assembly further defines an integrated case.

19. The stand according to claim 11 wherein the stand is fabricated from a waterproof or water-resistant material.

20. The stand according to claim 19 wherein the waterproof or water-resistant material is plastic.

21. The stand according to claim 19 wherein the waterproof or water-resistant material is stainless steel.

22. The stand according to claim 19 wherein the waterproof or water-resistant material is dishwasher safe.

23. A method for drying urinary catheters comprising vertically hanging one or more urinary catheters in the scallops of the shelf unit of the urinary catheter drying stand according to claim 11 and allowing the urinary catheters to dry.
